(19) United States
(12) Patent Application Publication
(10) Pub. No.: US 2008/0195099 A1
(43) Pub. Date: Aug. 14, 2008

(54) Osteotomy system

(75) Inventor: Tom Minas, Dover, MA (US)

Correspondence Address: Edwards Angell Palmer & Dodge LLP, P.O. Box 55874, Boston, MA, 02205, US

(73) Assignee: The Brigham and Women's Hospital, Inc., Boston, MA (US)

(21) Appl. No.: 11/999,287

(22) Filed: Dec. 5, 2007

Related U.S. Application Data
(60) Provisional application No. 60/901,039, filed on Feb. 13, 2007.

(51) Int. Cl. A61B 17/58; A61F 5/00; A61B 17/56

(52) U.S. Cl. 606/70; 606/87; 606/88; 606/301

(57) ABSTRACT

The invention provides a suite of instruments, implants and associated techniques for performing procedures to correct deformities of the knee of a patient. In accordance with a preferred embodiment of the invention, implants, a kit and associated methods are provided for correcting varus and valgus deformities of the knee.

### CROSS-REFERENCE TO RELATED APPLICATION

This application claims priority to U.S. Provisional Patent Application Ser. No. 60/901,039, filed Feb. 13, 2007. This patent application is incorporated by reference herein in its entirety.

### BACKGROUND OF THE INVENTION

1. Field of the Invention

The present invention relates to an improved method and system for performing osteotomy procedures. Particularly, the present invention is directed to a kit of instruments and associated techniques for performing corrections of various skeletal deformities.

2. Description of Related Art

Wedge osteotomy of the distal femur or proximal tibia is an increasingly important way to treat uni-compartmental arthritis of the knee joint. Using one of these procedures in appropriately selected patients can defer by several years the need for surgery to replace the entire knee joint with a prosthesis. Two thirds of patients with moderately advanced disease who undergo these procedures continue to do well up to ten years after surgery. The results are much better for patients with earlier stage disease, in which the articular surface is damaged, but there is no bone-on-bone contact between the femur and tibia. Under those circumstances, eighty percent of patients with earlier stage disease continue to do well up to 15 years after surgery. Weight and physical conditioning are important determinants of outcome; a patient whose weight exceeds 1.3 times his or her ideal body weight is much less likely to obtain good long term results.

Erosion of the articular surface of the medial compartment of the knee is associated with a varus deformity, in which the lower leg becomes angulated inward with respect to the long axis of the femur (a ‘bow-legged’ appearance). On the other hand, excessive wear of the articular surface of the lateral compartment of the knee is associated with a valgus deformity, in which the lower leg angles outward from the long axis of the femur (a “knock-knee'd” appearance).

Both of these effects alter the normal stress mechanics on the knee joint, and in fact accelerate the degeneration of the affected articular surface. Re-establishing or over-correcting the normal mechanical alignment of the lower leg with the femur and hip joint helps to off-load the affected compartment, and shifts weight-bearing to the less-affected compartment. This helps to delay progression of the disease and ultimately the need for prosthetic joint replacement surgery.

Correction of a varus deformity of the knee can be achieved by creating a wedge shaped angulation of the bone on either the medial side of the proximal tibia (a high tibial open wedge osteotomy), or the lateral side of the proximal tibia (closing wedge osteotomy). A high tibial opening wedge osteotomy, for example, involves making a cut in the proximal portion of the tibia approximately 1½ cm down from the joint surface (or at the level of the mid-fibular head), starting from its medial side and extending it sufficiently near the cortex of the bone on the lateral side to permit opening the wedge with a device. In an opening wedge osteotomy, the single cut on the medial proximal tibia allows a space to be created by forcing the cut surfaces of bone apart.

A similar procedure is used for a proximal tibial closing wedge osteotomy, with the exception that two cuts forming a wedge of bone are made on the lateral side, and the wedge of bone is removed. The first cut is made in a way similar to the open wedge osteotomy. The second cut is made a defined distance below the first cut, but angled so that the end of the cut meets the end of the first cut near the opposite cortex of the bone. This forms a wedge-shaped piece of bone that can then be removed. In a closing wedge osteotomy, the space created by removing the wedge of bone is closed by forcing the cut surfaces of bone together.

Importantly, the depth of the cut must be controlled so that it is not so close to the opposite cortex as to cause it to break when the wedge is either opened or closed. The amount of correction varies from about 5 degrees to about 20 degrees of wedge opening or closing, depending on the degree of deformity, the condition of the knee joint, the age and physical condition of the patient, among other factors. In either case, the result is a shifting of the mechanical axis laterally or medially on the tibia away from the affected compartment.

In an opening wedge osteotomy, once the correct opening width is obtained, a metal block of suitable thickness attached to a plate can be inserted into the defect. The plate bridges the defect, and is secured by screws into the intact bone on either side of the defect. Autologous bone graft fragments or biocomposite material, for example, are placed within the wedge defect. The biocomposite material is a synthetic absorbable calcium polymer composite, for example, that acts as a scaffold for new bone growth. Eventually, native bone cells migrate into, resorb and replace the autologous bone graft or biocomposite material. Healing times for open wedge osteotomies range between 3 and 4 months, depending on the degree of correction.

The healing time for a closing wedge osteotomy is much shorter, because the fusing surfaces of bone are placed into direct contact with each other. The defect is closed by pulling the extremities of the bone in the direction of the defect, bringing the two cut surfaces of bone together. A plate is then applied across the line of repair to hold the bone surfaces together. Because of the direct bone-to-bone healing, a closed wedge osteotomy generally takes only 6-8 weeks to heal.

Correction of a valgus deformity of the knee (a “knock-knee'd” deformity), on the other hand, can be accomplished by making the required corrections on the distal femur. Deterioration of the lateral compartment of the knee joint can occur, for example, in association with a hypoplastic lateral femoral condyle. Under these circumstances, the plane of the tibial plateau is sloped upward toward the lateral side. The goal of the osteotomy procedure in this case is to add bone to the lateral side of the distal femur through an opening wedge osteotomy on the lateral side, or remove bone from the medial side of the distal femur through a closing wedge osteotomy on the medial side. Either of these procedures results in a more horizontally oriented tibial plateau. The procedures are as described for the proximal tibia osteotomy procedures, with the exception that the shapes and dimensions of the plates differ in order to conform to the anatomical differences between the distal femur and the proximal tibia.

Several manufacturers produce and market devices for performing wedge osteotomies. Although each existing kit has some advantages, none combines the best technology to make the procedure as efficient and as reliable as possible. Currently, the procedure is not widely practiced by orthopedic surgeons for at least two reasons. First, no kit combines the best technology available to maximize the chance for success and minimize the risk of complications for all four procedures (involving the proximal tibia or distal femur on either the medial or lateral side). Second, no kit is complete enough to allow the surgeon to operate without first searching for and assembling additional instruments, wastefully opening several surgical kits to accomplish one surgical procedure. Moreover, even in combination, the instruments contained in these kits are not optimized for performing all four of the aforementioned procedures. The present invention provides a solution for these and other problems.

### SUMMARY OF THE INVENTION

The purpose and advantages of the present invention will be set forth in and become apparent from the description that follows. Additional advantages of the invention will be realized and attained by the methods and systems particularly pointed out in the written description hereof, as well as from the appended drawings.

To achieve these and other advantages and in accordance with the purpose of the invention, as embodied herein, the invention includes a multi-part implant for supporting an opening wedge osteotomy. The implant includes a spacer for insertion into an opening created during the osteotomy procedure. The spacer has a first contoured surface. The implant further includes a plate for spanning the opening created during the osteotomy procedure. The plate includes a first portion for attachment to bony tissue proximate a first side of the opening, a second portion for attachment to bony tissue proximate a second side of the opening, and a third portion for attachment to the spacer, the third portion of the plate including a second contoured surface that complements the first contoured surface to provide alignment between the spacer and plate when they are attached.

In accordance with a further aspect of the invention, the plate may define a length along a direction that spans an opening created during an osteotomy procedure and a width generally transverse to the length. The average width of the plate may be, for example, between about 1.5 cm and about 2.5 cm, among others. The plate preferably has a width sufficient to cover at least 50% of the width of the medial surface of the tibia. Even more preferably, the plate has a width sufficient to cover at least 60% of the width of the medial surface of the tibia.

In accordance with another aspect of the invention, the plate may include two rows of alternating holes along a majority of its length. If desired, the plate may further include a widened portion proximate an end of the plate adapted and configured to be attached to a head of a tibia. The plate may have a thickness between about two millimeters and about six millimeters. More preferably, the plate has a thickness between about three millimeters and about five millimeters. Most preferably, the plate has a thickness between about three and a half millimeters and about four and a half millimeters.

In accordance with a further aspect of the invention, the spacer may include at least two opposed bone engagement surfaces for engaging cortical bone created by an osteotomy. In accordance with one embodiment, the opposed bone engagement surfaces are substantially parallel. In accordance with another embodiment, the opposed bone engagement surfaces are tapered along an anterior-posterior direction. In accordance with this embodiment, the opposed bone engagement surfaces diverge along an anterior-posterior direction. If desired, the opposed bone engagement surfaces may converge along an anterior-posterior direction. The opposed bone engagement surfaces may taper with respect to each other at a number of suitable angles, such as about two degrees, about two and a half degrees, about three degrees, about three and a half degrees, about four degrees, about four and a half degrees, about five degrees, about five and a half degrees, and about six degrees, among others. Most preferably, the taper is about five degrees.

In accordance with still another aspect of the invention, the spacer may define an interior volume adapted and configured for receiving material therein. The spacer may further include material disposed in the interior volume to facilitate growth of bony tissue therethrough. The spacer is preferably shaped and sized to fill a substantial portion of an opening created during an opening wedge osteotomy procedure. For example, the spacer may be defined by an annular body surrounding a hollow core. By way of further example, the spacer may be defined by an annular body made from a first material surrounding a core made from a second material. For example, the first material may include a non-resorbable material and the second material may include a resorbable material. Preferably, the first material includes a material selected from the group consisting of titanium, aluminum, tantalum, a polymeric material, a composite material, and combinations thereof. Even more preferably, at least one of the first and second materials is sufficiently porous to permit the growth of bony tissue therethrough. The core may further define an opening through the center thereof sufficient to permit a stem portion of an implant to pass therethrough.

In still further accordance with the invention, the first and second portions of the plate may include protrusions to facilitate anchoring the plate to bony tissue of a patient. If desired, the first and second contoured surfaces may include at least one alignment feature for aligning the spacer with the plate. For example, the first and second contoured surfaces may comprise a dovetailed joint. In accordance with still another embodiment, the spacer may include a plurality of displaceable arms that anchor into adjacent bony tissue when a threaded connection between the spacer and plate is tightened.

In accordance with still a further aspect, a kit for performing an opening wedge osteotomy is provided. The kit includes a plurality of spacers for insertion into an opening created during the osteotomy procedure, each spacer having a first contoured surface. The kit further includes a plurality of plates for spanning the opening created during the osteotomy procedure. Each plate includes a first portion for attachment to bony tissue proximate a first side of the opening and a second portion for attachment to bony tissue proximate a second side of the opening. Each plate further includes a third portion for attachment to the spacer. The third portion of the plate includes a second contoured surface that complements the first contoured surface to provide alignment between the spacer and plate when they are attached.

In further accordance with the invention, the kit may include a plurality of fasteners, such as screws, for attaching the plates to bony tissue. Each fastener preferably includes at least one polished surface. If desired, each fastener may be provided with a portion that is adapted and configured to engage with the plate. For example, if a screw is used, it may include a head portion that engages with (e.g., locks with) the plate.

In accordance with still a further aspect of the invention, at least two of the spacers of the kit may be provided in different sizes. Moreover, at least two of the plates may be provided in different sizes. Preferably, at least one of the spacers is tapered along the anterior-posterior direction.

In accordance with still further aspects of the invention, the kit may further include at least one fastener for connecting a spacer to a plate. The kit may also include a cutting guide for attachment to bony tissue of a patient, the cutting guide defining a groove for receiving and guiding a cutting tool, such as a bone saw. If desired, an adjustable bone spreader may also be provided in the kit for spreading open a cut formed in bony tissue of a patient by a surgeon. Moreover, a retractor may also be provided in the kit for retracting a patient's patellar tendon. The patellar tendon retractor may include a pointed distal tip adapted and configured to be anchored into bony tissue to facilitate retraction of the patellar tendon. Furthermore, the kit may include at least one staple for implantation into bony tissue opposite an opening defined in a patient's bony tissue during an opening wedge osteotomy procedure.

In further accordance with the invention, a bone plate is provided for spanning a gap formed in a tibia in a patient subsequent to performing an opening wedge osteotomy. The plate includes a generally rectangular body having a length along a direction that spans the gap and a width generally transverse to the length. The average width of the plate is preferably between about 1.5 cm and about 2.5 cm.

In accordance with a further aspect of the invention, the plate is provided with a width sufficient to cover at least 50% of the width of the medial surface of a tibia of a patient. Even more preferably, the plate is provided with a width sufficient to cover at least 60% of the width of the medial surface of a tibia of a patient. If desired, the plate may includes two rows of alternating holes along a majority of the length of the plate. Preferably, the plate includes a widened portion proximate an end of the plate shaped for attachment to a head of a patient's tibia.

In accordance with a further aspect of the invention, first and second portions of the bone plate may include protrusions for anchoring the plate to bony tissue of a patient. If desired, the plate may further include a spacer disposed on an anatomically-facing surface for maintaining an open wedge osteotomy. Preferably, the spacer and plate are removably attached to each other by a fastener. Most preferably, each of the spacer and plate include cooperating alignment features for maintaining registration between the spacer and plate.

It is to be understood that both the foregoing general description and the following detailed description are exemplary and are intended to provide further explanation of the invention.

The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIGS. 1(a) and 1(b) are front schematic views illustrating certain bones and tendons of a knee of a patient undergoing a surgical procedure to correct a deformity of the knee using patellar tendon retractors made in accordance with the present invention.

FIG. 2 is a top view of a knee joint (showing the top of the tibia) illustrating the bones and tendons of a knee of a patient undergoing a surgical procedure to correct a deformity of a knee using the patellar retractor illustrated in FIG. 1, as well as a posterior tibial or femoral retractor made in accordance with the present invention.

FIG. 3 is a side view of a breakaway bone pin made in accordance with the present invention.

FIGS. 4(a)-4(c) depict end and side views, respectively, of cutting guide blocks made in accordance with the present invention.

FIGS. 5(a)-5(f) schematically depict the placement of a cutting guide block and bar assembly for a closing wedge osteotomy made in accordance with the present invention.

FIGS. 6(a)-6(c) are schematic views of a bone spreader made in accordance with the present invention.

FIGS. 7(a)-7(b) are views of a right leg medial tibial wedge plate depicted in association with the anatomy of a patient's knee and a left medial tibial wedge plate made in accordance with the present invention.

FIG. 8 is an isometric view of a front face of an opening wedge plate made in accordance with the present invention.

FIG. 9 is an isometric view of a back face of an opening wedge plate made in accordance with the present invention illustrating a tenon portion of the plate.

FIG. 10 is a cross sectional view of an opening wedge plate and associated spacer made in accordance with the present invention, illustrating the mating of a tenon of the opening wedge plate, and a mortise of the spacer.

FIGS. 11(a)-11(f) are schematic views of an opening wedge plate and associated spacers made in accordance with the invention depicting the placement of the same in the tibia of a patient.

FIG. 12 schematically depicts a femoral opening wedge plate made in accordance with the present invention in relation to a patient's skeletal anatomy.

FIG. 13 schematically depicts a femoral opening wedge plate made in accordance with the present invention and a femoral closing wedge plate made in accordance with the present invention in relation to a patient's skeletal anatomy.

FIGS. 14-15 depict front and side views of an exemplary bone staple for use in association with other components of the invention.

FIG. 16 depicts the staple depicted in FIGS. 14-15 mounted in the distal end of an insertion tool.

FIG. 17 depicts a kit for inserting and extracting staples.

### DETAILED DESCRIPTION OF THE PREFERRED EMBODIMENTS

Reference will now be made in detail to the present preferred embodiments of the invention, examples of which are illustrated in the accompanying drawings. The method and corresponding steps of the invention will be described in conjunction with the detailed description of the system and kit.

The devices, methods and kits presented herein may be used for performing osteotomy procedures. Generally, the systems and methods illustrated herein provide for more reliable placement of instrumentation to facilitate the modification of the skeletal structure. In particular, the instrumentation illustrated herein is well-suited, particularly in combination, for reliably and accurately removing wedge shaped pieces of bone from an elongate major bone of the body in order to shorten that side of the bone. The instrumentation is also well-suited for creating wedge-shaped spaces in such skeletal structures, which may then be packed with bone morphogenic material to stimulate the growth of bony tissue into these structures to, in effect, “create” a lengthening of that side of the bone.

The present invention is particularly well suited for surgical procedures that compensate for varus and valgus deformities of the knee. These procedures and associated instrumentation can be used to modify the geometry of the knee joint of a patient by modifying the distal (i.e., lower) end of the femur and/or the proximal (i.e., upper) end of the tibia in a variety of ways. It will be understood that the particular use illustrated herein is not limiting, and that the systems depicted herein may be modified, as appropriate, to perform similar procedures (of removing wedge shaped pieces of bone or inducing bone growth in wedge shaped spaces in bone) wherever desired in the skeletal anatomy of humans, as well as in veterinary applications, if desired.

Accordingly, and in accordance with an exemplary aspect of the invention, a system of instrumentation and associated methods are provided for correcting varus and valgus deformities of the knee. The system may include a variety of retractors adapted to move particular anatomy (e.g., tendons) out of the surgical area and to otherwise protect them from harm during the procedure. The system also may include a variety of guide blocks for guiding placement of a cutting instrument (such as a saw) to reliably and predictably cut bony tissue in a desired manner. The guide blocks may be held in place with respect to the patient's anatomy using guide pins made in accordance with the subject invention and/or other suitable fasteners. After a desired cut is made in the patient's anatomy, a wedge shaped opening may be created in the bone by inserting a bone spreader into the cut, and expanding the cut outward using the bone spreader to create a wedge shaped opening. If desired, a side of the bone opposite the opening may be reinforced with a fastener, such as a staple, to prevent the bone from separating proximate the cut. This opening may be maintained by securing a bone plate to the skeletal structure on either side of the opening (with or without a detachable block), and material can be inserted into the opening to stimulate bony growth into the opening.

In accordance with another embodiment of the invention, if desired, a wedge shaped piece of bone can be removed from the bone to create a wedge shaped opening, which may then be closed by bringing two faces of the opening together. The faces may be held together by securing bony material on either side of the opening with a bone plate. If desired, a side of the bone opposite the opening may be reinforced with a fastener, such as a staple, in order to prevent the bone from separating. Particular instrumentation that may be used to carry out these and other procedures, as desired, is discussed in detail below.

To begin a procedure as embodied herein, a surgical opening is made to access a patient's knee. It is particularly advantageous for the soft tissues adjacent to the knee joint to be properly retracted to avoid being harmed by the drilling and sawing of bone that procedures in accordance with the invention require. For example, the popliteal artery in the posterior aspect of the knee joint, and the patellar tendon anteriorly are particular structures in need of such protection. Accordingly, in accordance with the invention, a system is provided including retractors that are specially designed for each of these locations.

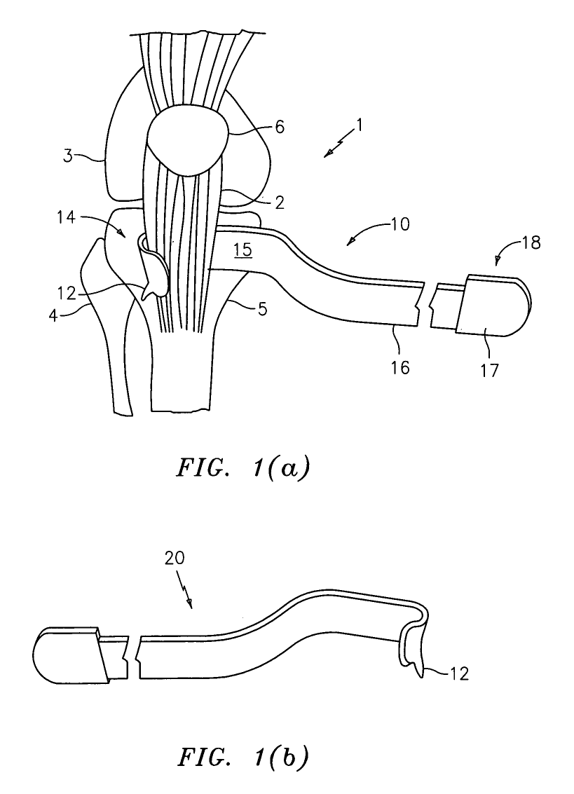

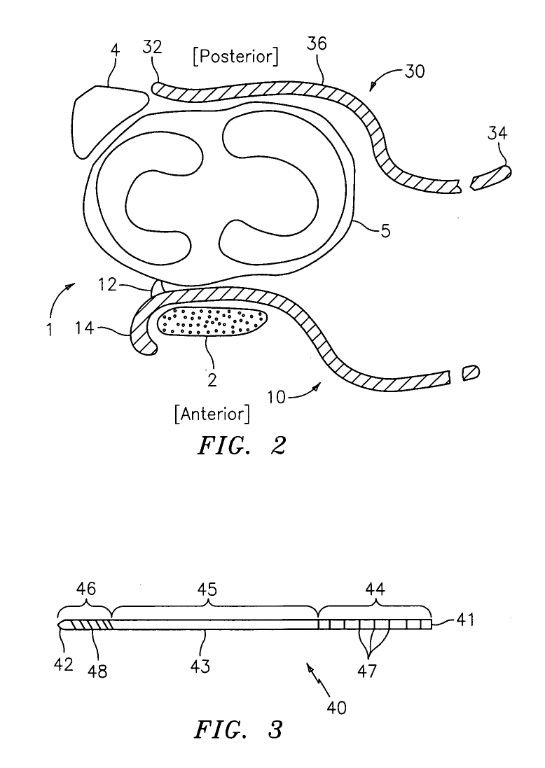

For purposes of illustration and not limitation, as embodied herein and as depicted in FIGS. 1-2, a schematic representation of a patient's anatomy proximate the knee 1 is presented. In particular, the distal (lower) end 3 of the femur is presented, as well as the proximal ends of the tibia 5 and fibula 4. The patellar tendon 2 is also presented, which attaches to the tibia 5 and patella 6. Also depicted in FIG. 1 is a novel retractor 10 for surrounding the patellar tendon 2 to protect the tendon 2 and move it, as desired. Retractor 10 includes a pointed distal tip 12 proximate a distal region 14 of retractor 10 adapted and configured to surround and protect the patellar tendon 2.

As depicted in FIG. 1, retractor 10 wraps around the patellar tendon 2, providing for a flat protective surface 15 on the posterior side of the tendon (adjacent to the anterior surface of the tibia). Retractor 10 can be about 1-2 centimeters, or preferably about 1-1.5 centimeters in width, as appropriate for the particular patient's anatomy. Retractor 10 wraps around the opposite side of the tendon 2, and a distal tip 12 projects from an area near the inferior margin of the distal region 14 of retractor 10. The pointed tip 12 of retractor 10 allows a surgeon to dig tip 12 into the bone to form a fulcrum point at tip 12 and obtain purchase on the surface of the tibia 5 to thereby retract the tendon 2 forward and away from the operative field by manipulating retractor 10, helping to avoid the risk of injury to the tendon 2 from the drilling and cutting of the bone. Retractor 10 further includes an elongate shaft 16 connecting the distal region 14 of the retractor to a proximal end 18 of the retractor, which includes a handle 17. Once the retractor 10 is properly anchored, the amount of force that a surgical assistant needs to apply to retract the tendon 2 is reduced and the surface of the tendon 2 is accordingly protected.

Retractor 10 can be a ‘z-type’ retractor that has been modified to have the proper geometry as described herein. Retractor 10 depicted in FIG. 1(a) is suitable for use when access to the medial side of the right knee 1 or lateral side of the left knee is desired. Retractor 20 in FIG. 1(b) is a mirror image version of retractor 10, suitable for use when access to the lateral side of the right knee 1, or medial side of the left knee is desired. Retractors 10 and 20 are preferably radiolucent (avoiding interference with x-rays or fluoroscopy during the procedure). The cross-sectional outline of the retractor 10 is also depicted in FIG. 2, in which a transverse plane through the tibial plateau is viewed from a superior orientation.

As further depicted in FIG. 2, a posterior retractor 30 is provided for retracting and protecting the popliteal artery and other soft tissues in the posterior aspect of the knee joint. Retractor 30 includes a proximal end 34, a distal end 32 and has an elongate body 36 that conforms to the contour of the posterior aspect of the knee joint. For example, retractor 30 may be a modified version of an existing retractor (such as an ‘Army-Navy’ retractor) made to conform to the contour of the posterior aspect of the knee joint to retract the popliteal artery. Specifically, retractor 30 is preferably angled to conform to the contour of the posterior aspect of the proximal tibia or distal femur. Retractor 30 is positioned between the tibia 5 and the adjacent soft tissues, the most important of which is the popliteal artery (not shown). Retractor 30 is adapted and configured to protect these soft tissues from injury during drilling and cutting of the bone. Retractor 30 can be about 1-2 centimeters, or preferably about 1.5-2 centimeters in width, as appropriate, depending on the particular patient's anatomy; and it has an approximate ‘S’ shape when viewed from the side.

Certain procedures performed in accordance with the invention utilize the placement of guide elements such as guide pins through the bone. Accordingly, the placement of such guide elements should be reliable and consistent. Once in place, these elements guide a cutting member (such as a saw blade) to make desired cut(s) through the bone. Accordingly, in further accordance with the invention, guide elements, such as guide pins are provided for permitting precise cuts through the bone, and for reliable placement of guide blocks to facilitate accurate cutting of bone.

For purposes of illustration and not limitation, as embodied herein and as depicted in FIG. 3, a guide pin 40 is provided. As depicted, guide pin includes a proximal end 41, a distal end 42, and an elongate body 43. Body 43 includes a proximal region 44, a middle region 45 and a distal region 46.

Designs for pins heretofore known in the art allow the pins to be placed in the chuck of a drill, for example. Cutting tips provided on the tip of the pins allow them to be drilled in the proper direction and to the proper depth of a bony structure of a patient. However, such pins known in the art currently used for osteotomies can loosen and dislodge from the vibrations induced by the saw.

In contrast, pin 40 includes self-tapping threading 48 in the distal region 46 of pin 40 to prevent backout and/or dislodging of pin 40 after it has been installed. As such, once the pin 40 has cut its way to the selected depth in the bone, the threads 48 reliably secure the pin 40 in place, avoiding any vibration-induced dislodgement. Pin 40 can also included breakaway features along the proximal region 44 of the body 43 of the pin 40 to allow the surgeon to quickly and efficiently break off the pin 40 near the surface of the bone once it has been properly placed. As depicted in FIG. 3, pin 40 includes a plurality of breakaway regions 47, that can be snapped off by a surgeon using a cutting tool (e.g., clippers) along a desired mark 47. Mark 47 can take on a variety of forms, but preferably includes at least an indentation formed into the surface of the pin 40 to permit the cutting jaws of a suitably configured cutting instrument to register therewith, to permit pin 40 to cut to the proper length.

In further accordance with the invention, cutting guide blocks are provided for facilitating accurately placed cuts through the bone of a patient.

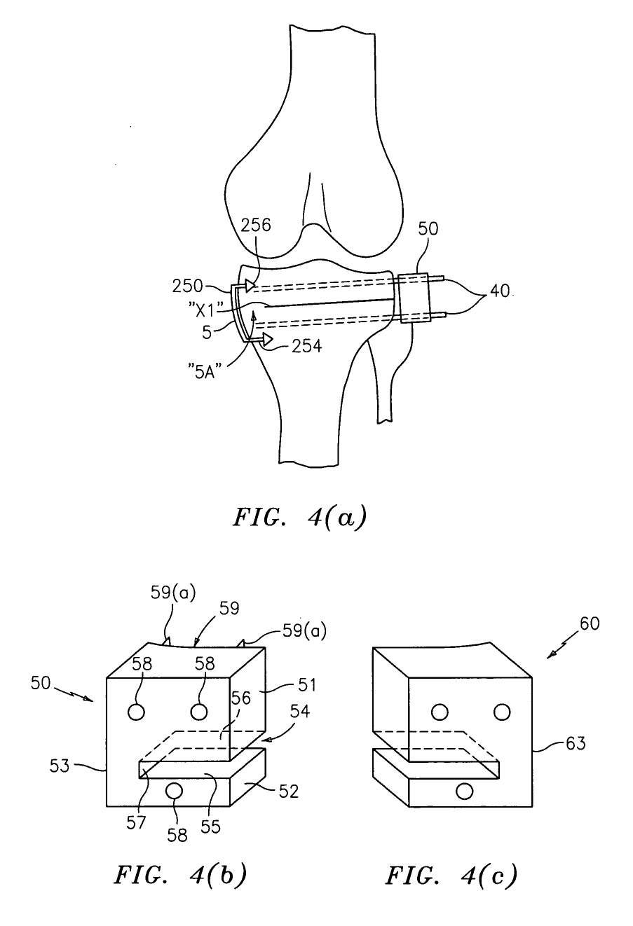

For purposes of illustration and not limitation, as embodied herein and as depicted, for example, in FIGS. 4(a)-4(b), cutting guide block 50 is are provided for a left lateral tibial osteotomy, or a right medial tibial osteotomy. As depicted, cutting guide block 50 includes a first portion 51 and a second portion 52 joined by a bridge portion 53. Portions 51-53 cooperate to define a groove 54 for receiving a cutting instrument, such as a bone saw. Specifically, groove 54 is defined by opposing faces 55-56 and end face 57 of block 50. Holes 58 are provided for receiving guide pins 40. Guide block 50 is secured to the bone of the patient, such as tibia 5 as depicted in FIG. 4(a) by way of guide pins 40, which may in turn be installed by a drill (not shown). As depicted, guide block 50 includes three holes 58 through which breakaway pins 40 are drilled into the lateral left tibia in the case of a tibial closing wedge osteotomy (or lateral left femur, in the case of a femoral opening wedge osteotomy). In one embodiment, two of the holes and breakaway pins are positioned above the cut site, and one is positioned below the cut site. If desired, block 50 may be provided with a contoured bone facing surface 59 for providing an improved fit with the patient's anatomy. By way of further example, if desired, one or more anchoring elements, such as barbs 59(a), may be provided proximate surface 59 to penetrate the bone and to help hold block 50 stationary with respect to the bone while the guides (e.g., pins) are inserted.

In accordance with another embodiment, cutting guide block 60 is provided for use on the right lateral tibia and left medial tibia, as shown in FIG. 4(c). In a further embodiment, guide block 50 can also be used on the right medial femur and left lateral femur, and guide block 60 can also be used on the right lateral femur and left medial femur. Although a preferred embodiment includes a guide block 50 or 60 whose bridge portion 53 or 63 faces anteriorly, it will be apparent to one skilled in the art that a guide block can also be constructed to have its bridge portion facing posteriorly.

After guide block 50 is secured into place, a first cut “X1” can be made into the bone of the patient to a desired depth determined by the surgeon. The desired depth of the cut can be determined by using the guide pins 40 as a reference point. Specifically, if the guide pins are radiopaque, they can be placed under fluoroscopy by the surgeon at a desired angle and depth. Markings 47 along the guide pin 40 can indicate the depth of the guide pins, and be used to select a depth for cutting the bone from the lateral side of the knee, as depicted in FIG. 4(a). Using the guide pins 40 as a reference point to control the depth of the cut can help prevent the cut from being made too deeply, which could undesirably compromise the structural integrity of the bone. Retractors 10 and 30 similarly provide hard surfaces against which a saw blade can contact when making cut “X1”. This helps ensure that the bone is cut all the way through along an anterior-posterior direction, yet ensure that the cut is not too deep into the bone along a lateral-medial direction.

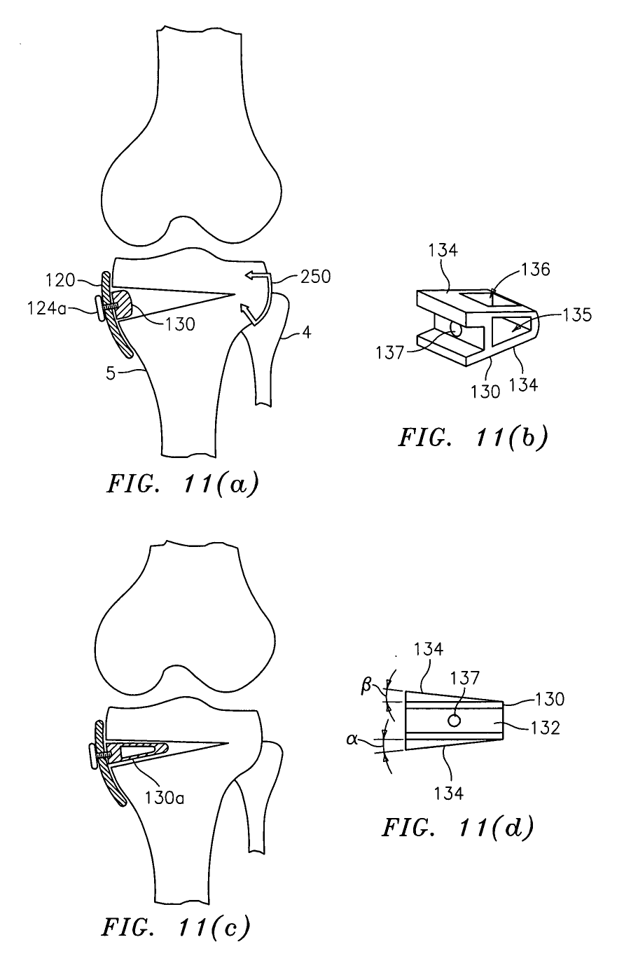

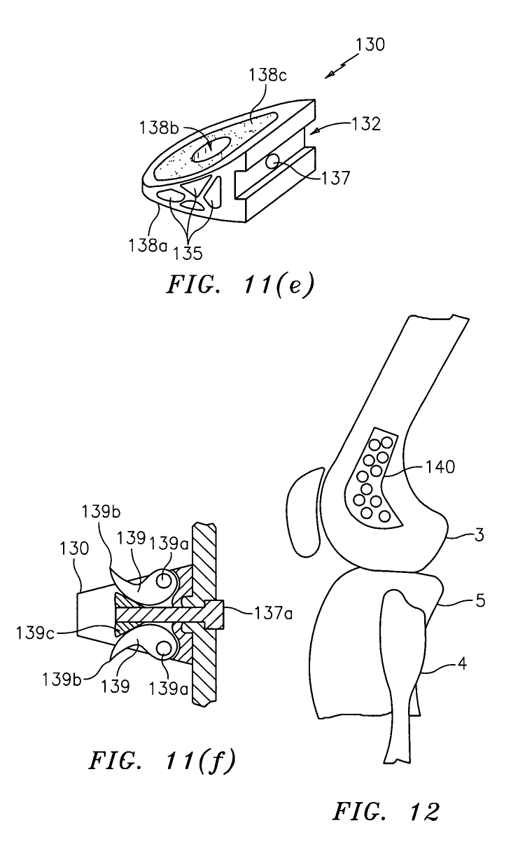

To help prevent the uncut portion of the bone “5A” from fracturing, or to prevent separation of the upper and lower portions of the bone 5 in the event of fracture, one or more fasteners such as staple 250 may be inserted in the bone 5 with a first prong 254 of the staple being anchored into the portion of the bone above the cut, and second prong 254 of the staple 250 being inserted below the cut. A further example of placement of a staple is depicted in FIG. 11(A). Staple 250 and associated instrumentation are described in further detail below.

Once the first cut X1 has been made, depending on the procedure, a second cut may be made as described in detail below in the case of a “closing wedge” osteotomy. In accordance with another aspect of the invention a bone spreader may be employed after a first cut is made to perform an “open wedge” osteotomy, although it will be recognized that an opening wedge procedure will be performed from the medial side of the proximal tibia, or the lateral side of the distal femur.

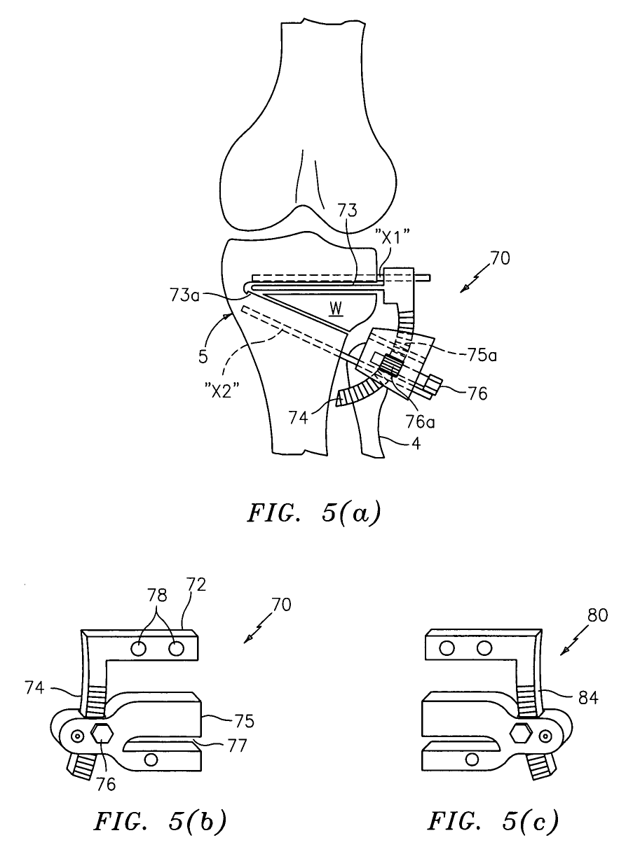

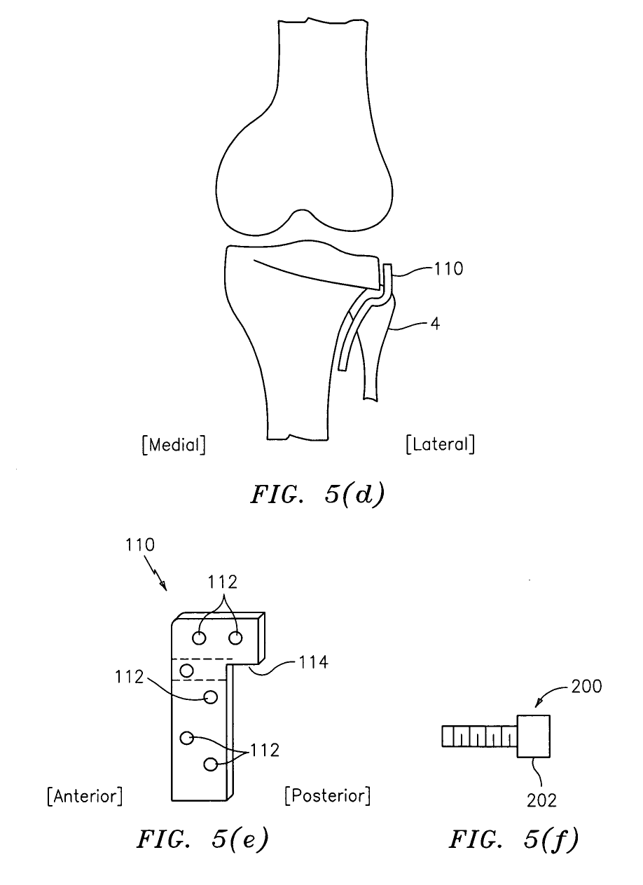

Thus, in accordance with one embodiment of the invention, a system and method for performing a “closing wedge” osteotomy is provided on the tibia or the femur. When a closing wedge osteotomy is performed on the tibia, access is had to the knee from the lateral (outside) face of the knee, as depicted in FIGS. 4-5.

For purposes of illustration and not limitation, to perform a “closing wedge” osteotomy on the left lateral tibia or the right medial femur, a first cut is provided through a patient's bone, such as the cut “X1” as described above with reference to FIG. 4(a). Next, as shown in FIG. 5(a), a second cut “X2” is provided along a second plane that is oriented at an angle with respect to the plane in which the first cut “X1” lies.

As embodied herein and as depicted in FIGS. 5(a)-5(e), a guide bar-and-block mechanism 70 is provided that fixes and guides the desired angle of the second cut “X2” and therefore the size of the wedge of bone to be removed.

Mechanism 70 includes a stationary portion 72 having two holes 78 that are adapted and configured to mate with the guide pins 40 that were originally installed to align guide blocks 50. Stationary portion also has an elongate planar tongue portion 73 adapted to be slid into substantially the full depth of cut “X1”, as depicted in FIG. 5(a). Stationary portion 72 further includes an arcuate guide rail 74 on which a movable portion 75 of mechanism 70 is movably mounted.

Movable portion 75 of mechanism 70 includes an arcuate passage 75a adapted to receive rail 74. Preferably, rail 74 and passage 75a each have a matching cross section defined by a plurality of sides, to prevent rotation between components 72, 75, but permitting angular translation afforded by the curvature of rail 74 between components 72, 75. The relative angular position of components 72, 75 may be selectively fixed by tightening a set screw 76. If desired, rail 74 can be provided with a rack gear and the set screw 76 can be provided with an adjustment mechanism including a pinion 76a to engage rack on rail 74 to form a rack and pinion adjustment that can provide continuous or ratcheted angular adjustment in any desired amount, such as in increments of one degree. Preferably, the center of an arc defined by rail 74 substantially coincides with the leading edge 73a of tongue 73. Movable portion 75 bears certain similarities to guide block 50, in that it defines a similar groove 77 for guiding a saw blade for making second cut “X2”.

In use, guide block 50 is removed after having performed cut “X1”, leaving guide pins 40 in place. The lowermost, third guide pin 40 is removed from the bone. Next, stationary portion 72 of mechanism 70 is installed over the remaining guide pins 40. The angular displacement of portion 75 is adjusted with respect to stationary portion 72 until a desired angle has been defined for second cut “X2” with respect to cut “X1”. Portion 75 is then set in place with setscrew 76 and another breakaway pin 40. Cut “X2” is then made through the bone, liberating a wedge-shaped piece of bone “W”. This piece of bone W is then removed.

After the wedge of bone is removed, the pins 40 are removed, and the remaining portions of the bone are then brought together and held in place by a bone plate.

In another embodiment, guide bar and block mechanism 80 is provided for use on the right lateral tibia and left medial femur, as shown in FIG. 5(c). Although a preferred embodiment includes a guide block 70 or 80 whose rail portion 74 or 84 faces anteriorly, it will be apparent to one skilled in the art that a guide bar and block can also be constructed to have its rail portion facing posteriorly.

In accordance with one aspect of the invention for a closing left lateral tibial osteotomy, as embodied herein and as depicted in FIGS. 5(d)-5(f), a closing tibial osteotomy plate 110 is provided. Plate 110 is preferably “L-shaped”, with an indented portion 114 on its right side as shown in FIG. 5(E) to avoid interfering with the proximal left fibula 4, and uses locking screws 200 having threaded heads 202 that lock into threaded holes 112 in the locking plate 110 to enhance the rigidity of fixation. Preferably, the vertical cross-sectional profile of the plate 110 is offset as shown in FIG. 5(d) to more closely match the contour of the lateral aspect of the proximal tibia, and the offset that results from re-approximating the cut surfaces of bone. Several variations of plate 110 can be provided, each with a different degree of offset, ranging from about 1 mm to about 10 mm or more. Plate 110 can be made from a material such as stainless steel that, while rigid, can be bent by a surgeon during a surgical procedure to more closely conform to the patient's anatomy. It will be understood that any suitable number of holes 112 can be provided in plate 110. Plate 110 can also be made from a material such as titanium, which can be stronger, less likely to interfere with a magnetic resonance imaging (MRI) scan, and, when in a polished form, allow for easier removal of the fixation screws. Plate 110 can also be constructed so that its indented portion 114 is situated on its left side (as viewed in FIG. 5(e)) to permit its installation on the right lateral tibia.

Moreover, as will be appreciated by those of skill in the art, any bone plate disclosed herein requiring mounting in bone with a fastener may use any of a variety of techniques and mechanisms to prevent the backout of bone screws. For example, suitable mechanisms are described in U.S. Pat. No. 6,331,179, U.S. Pat. No. 6,383,186 and U.S. Pat. No. 6,428,542. Each of these patents is incorporated by reference herein in its entirety.

In certain circumstances, it may be desirable to perform an “open wedge” osteotomy on the tibia or the femur, as desired. On the tibia, an open wedge procedure is generally performed from the medial side of the knee, whereas on the femur, an open wedge osteotomy is generally performed on the lateral side of the knee.

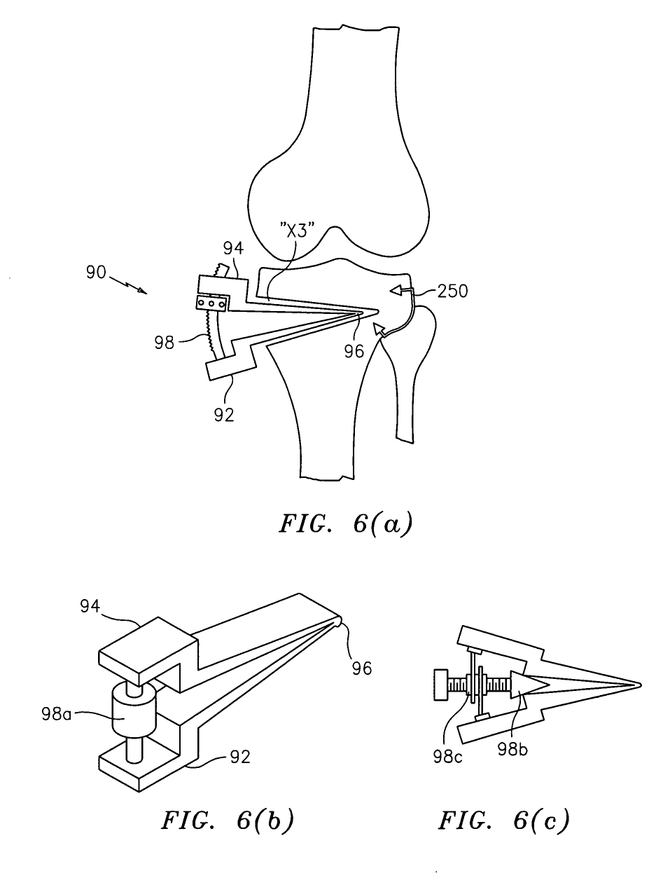

For purposes of illustration and not limitation, as embodied herein and as depicted in FIG. 6(a), a portion of an open wedge osteotomy procedure is depicted. Initially, a cutting guide block, similar to guide block 60, but adapted to engage the tibia from the medial side is attached to the medial side of the tibia by way of three guide pins 40, and a cut “X3” is made through the tibia along an anterior-posterior direction, but stopping short of the lateral cortex of the tibia. To create an “open wedge”, a bone spreader 90 is placed into the cut “X3”, and expanded to create an open wedge-shaped space in the bone, effectively using the remaining bone of the tibia as a “hinge”. As depicted, spreader 90 includes a first portion 92 connected to a second portion 94 via a hinge portion 96. It will be understood that hinge 96 can be a pivot point that joins portions 92, and 94, or may be a “living hinge” whereby portions 92, 94 are formed from the same piece of material. Spreader 90 further includes an expansion mechanism 98 joining the non hinged ends of portions 92, 94. Expansion mechanism is used to splay apart portions 92, 94 to create the wedge-shaped space.

Expansion mechanism 98 can take on a variety of forms. While expansion mechanism is depicted as a ratchet or rack and pinion mechanism, other mechanisms may be used, such as a hydraulic mechanism 98a (FIG. 6(b)), or a wedge block 98b that is urged toward the hinge by rotation of a threaded drive 98c as depicted in FIG. 6(c).

The bone spreaders depicted herein combine the advantage that a solid metal wedge provides (obtaining an accurate and secure wedge opening) with the safety of using a conventional bone spreader that does not need to be pounded into the bone cut. Specifically, existing solid metal wedges known in the art are marked to assist the surgeon in keeping from opening the bone wedge too widely. However, pounding these wedges in with a hammer risks fracturing the opposite cortex of the bone, creating a complete osteotomy and significantly complicating the procedure. Such a risk is less likely with a traditional bone spreader, but this device requires significant force and does not allow for a precise wedge opening. Other bone spreaders such as the Synthes TomoFix™ Bone Spreader allow for screw-driven wedge opening, but the point of engagement of the screw is awkwardly situated on the inferior or superior surface of device. Accordingly, any of the bone spreaders provided by the present invention is thin enough to be slid into the bone cut, and is controlled by an expansion mechanism that opens the spreader 90 in precise increments using an engagement mechanism located on the lateral (outer) aspect of the device, where it is easily accessible. The spreader 90 can be opened by gradations, and/or continuously, and can have an engraved or printed scale allowing the surgeon to accurately control the degree of wedge opening in increments of less than 1 degree, and to an opening of 20 degrees or more.

Once a wedge shaped opening has been formed in the bone of a patient, the space can be secured and packed with bone generating material to fill the void. Accordingly, in further accordance with the invention, a plate and spacer system is provided that maintains the proper gap in the opening wedge osteotomy. The plates may be provided with an L-shape that allows a superior aspect of the plate to be secured to a longer anterior-posterior segment of the proximal tibia above the wedge incision. The plate is attached in a more forward position but onto a longer superior-inferior segment of the tibia below the wedge incision. This keeps the plate from interfering with the attachment of the pes anserinus tendons on the proximal tibia.

The system of the invention provides a set of spacers of different sizes to meet the needs of the particular case. These spacers, unlike existing devices, can be attachable to the plates in the kit by means, for example of one or more fasteners such as screws and a rigid connection, such as a mortise-and-tenon connection, dovetailed connection or other connection.

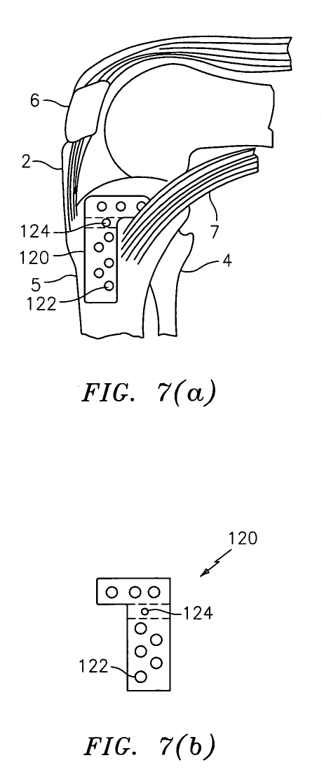

For purposes of illustration and not limitation, as embodied herein, opening wedge plates 120 designed for example, for the medial aspect of the proximal tibia are illustrated in FIGS. 7(a)-7(b). The L-shape allows the plate 120 to avoid interfering with the insertion of the pes anserinus tendons 7. The plate 120 is relatively short in its proximal-distal dimension to minimize the dissection required for the procedure. It is sufficiently wide to allow for two columns of staggered screw holes 122, as shown in FIGS. 7-9.

The plate 120 includes a first portion 120a for attachment to bony tissue proximate a first side of the opening, a second portion 120b for attachment to bony tissue proximate a second side of the opening, and a third portion 126 for attachment to spacer 130. Third portion 126 of the plate 120 includes a contoured surface 120c that complements a contoured surface 130c on the spacer 130 to provide alignment between the spacer 130 and plate 120 when they are attached to each other. The first and second portions of the plate may include protrusions 120e to facilitate anchoring the plate to bony tissue of a patient.

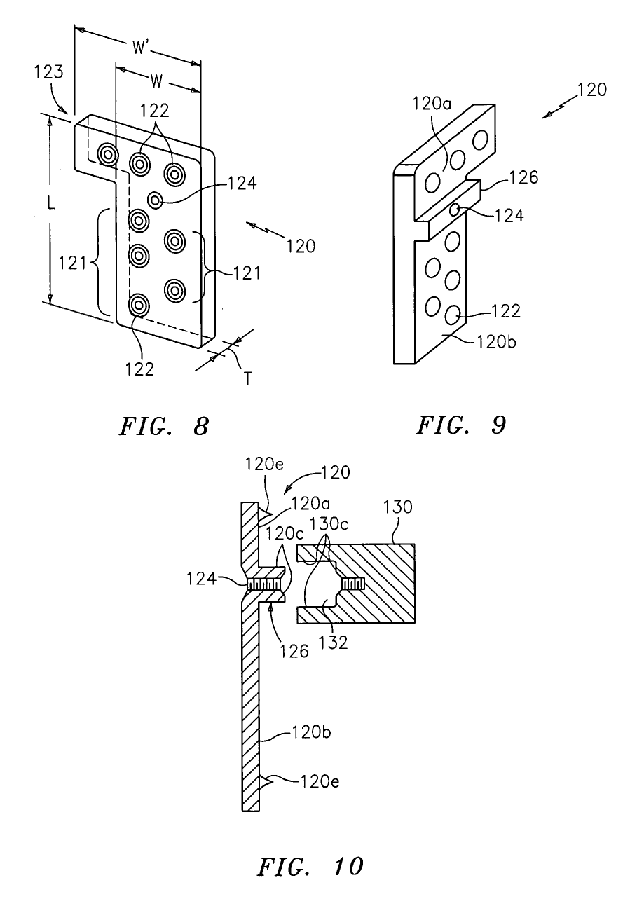

As further depicted in FIGS. 8-9, plate 120 may define a length L along a direction that spans an opening created during an osteotomy procedure and a width W generally transverse to the length. The average width W of the plate may be, for example, between about 1.5 cm and about 2.5 cm, among others. The plate preferably has a width sufficient to cover at least 50% of the width of the medial surface of the tibia. Even more preferably, the plate has a width sufficient to cover at least 60% of the width of the medial surface of the tibia.

In accordance with another aspect of the invention, the plate may include two rows 121 of alternating holes 122 along a majority of its length. If desired, the plate 120 may further include a widened portion 123 proximate an end of the plate adapted and configured to be attached to a head of a tibia, providing for an increased width W′ proximate an end of the plate 120. The plate 120 may have a thickness T, for example, between about two millimeters and about six millimeters. More preferably, the plate 120 has a thickness T between about three millimeters and about five millimeters. Most preferably, the plate 120 has a thickness T between about three and a half millimeters and about four and a half millimeters.

As depicted, the holes 122 of plates 120 may be threaded and tapered to accept locking screws 200 that have threaded heads as described above. Another screw hole 124 is present opposite the site of a tenon 126 on the side of the plate that faces the bone, as shown in FIG. 9. The interlocking arrangement between the first contoured surface (in this case mortise 132) of spacer 130 and the second contoured surface (in this case tenon 126) of the associated plate 120 is shown in the cutaway view of the locking plate and spacer system as depicted in FIG. 10. Although a mortise and tenon coupling is depicted in the aforementioned illustrations, it will be apparent to one skilled in the art that other alignment and coupling mechanisms between the plate 120 and spacer 130 are possible as mentioned herein above. In general, however, it is advantageous to have a coupling or interlocking system that allows for rapid positive alignment of the screw hole 124 of the plate with the corresponding attachment screw hole of the spacer 130, and that inhibits rotation or translation of the spacer 130 with respect to the plate 120.

An interesting feature of the detachable plate and spacer system of the depicted embodiments is the interchangeability of different size spacers 130 with any given plate 120. This feature reduces the number of components that must be included in an osteotomy kit, and increases the efficiency with which a surgeon can select and customize a plate-spacer combination to the needs of the patient during surgery.

As shown in FIGS. 11(a) and 11(c), the spacer 130 functions to maintain the proper gap in the osteotomy wedge while the plate 120 is being affixed to the bone 5. The plate 120 may be affixed by first drilling into the bone through the screw holes 122 on the plate 120, and then placing the screws 200 into the bone through the holes on the plate. If desired, the screws 200 may be configured to lock onto the plate by means of threaded locking heads 202 that screw into corresponding threaded recessed holes in the plates as described herein. This fixation method prevents any movement between the screws 200 and the plate 120, which reduces the chances of metal fatigue and ultimately possible fracture of the screws near the plate. Preferably, the fixation of the locking head screws to the plate is sufficiently secure such that maintaining the spacer within the osteotomy wedge after fixation of the plate is optional.

The affixed wedge plate 120 and spacer 130 are shown schematically in FIGS. 11(a) and 11(c) in situ in an open wedge osteotomy space. After the locking screws 200 and plate 120 are firmly anchored into the bone 5, the spacer 130 may optionally be removed by unscrewing the set screw 124a from threaded hole 137 and sliding the spacer 130 out. The space that remains within the open wedge may then be filled with bone graft fragments or synthetic biopolymer material, as desired.

Removing the spacer after placement of the locking plates can be advantageous, as this permits the entire osteotomy wedge to be filled with bone graft or a synthetic biocomposite material, which may increase the speed of healing. Alternatively, depending on the patient and the condition of the bone, it may be desirable to keep the spacers in place during recovery to provide additional stabilization of the osteotomy wedge during weight-bearing activity. Another alternative is to provide the spacer 130 made at least in part from a resorbable material that can be machined into the shape of a wedge.

Spacer 130 may define an interior volume adapted and configured for receiving material therein. For example, as depicted in FIG. 11(b), spacer 130 can define a plurality of openings 135 within it to define an interior volume 136 that can be packed with autologous bone graft, biocomposite material, and/or bone morphogenic protein (“BMP”) to stimulate bone growth. Spacer 130 may be shaped and sized to fill a substantial portion of an opening created during an opening wedge osteotomy procedure as depicted in FIG. 11(c). For example, as depicted in FIG. 11(e), spacer 130 may be defined in part by an annular body 138a surrounding a hollow core 138b. By way of further example, the spacer 130 may be defined by an annular body 138 made from a first material surrounding a core 138c made from a second material. For example, the first material may include a non-resorbable material and the second material may include a resorbable material. Preferably, the first material includes a material selected from the group consisting of titanium, aluminum, tantalum, a polymeric material, a composite material, and combinations thereof. Even more preferably, at least one of the first and second materials is sufficiently porous to permit the growth of bony tissue therethrough. The core may further define an opening 138b through the center thereof sufficient to permit a stem portion of an implant to pass therethrough during a later total knee replacement procedure. Providing such an opening eliminates the need to remove the spacer 130 during such a procedure.

Maintaining a spacer 130 in place after a procedure rather than removing it can be advantageous since it can bear weight without being forced out of the wedge. It is advantageous to bear weight on a bone graft while it is healing, since in accordance with Wolff's law, bone growth occurs most effectively under loading. If desired, the spacer 130 can be made from titanium or stainless steel. The wedge can also be made of tantalum, which has been shown to have greater porosity, reduced stiffness and a higher friction coefficient that titanium alone—properties that are conducive to enhancing the ingrowth of bone.

As depicted, spacer 130 includes at least two opposed bone engagement surfaces 134 for engaging cortical bone created by an osteotomy. In accordance with one embodiment, the opposed bone engagement surfaces 134 are substantially parallel. In accordance with another embodiment, as depicted in FIG. 11(d), the opposed bone engagement surfaces 134 are tapered along an anterior-posterior direction (or a posterior-anterior direction). In accordance with this embodiment, the opposed bone engagement surfaces 134 diverge or converge along an anterior-posterior direction. As depicted in FIG. 11(d), each surface 134 may be tapered with respect to the centerline of the spacer 130 by a predetermined angle (α, β). Alternatively, only one surface 134 may be inclined and the other surface 134 may be parallel to the centerline of the implant. The opposed bone engagement surfaces 134 may taper with respect to each other at a number of suitable angles, such as about two degrees, about two and a half degrees, about three degrees, about three and a half degrees, about four degrees, about four and a half degrees, about five degrees, about five and a half degrees, and about six degrees, among others. Most preferably, the total taper is about five degrees.

In accordance with still another embodiment, the spacer 130 may include a plurality of displaceable arms 139 that anchor into adjacent bony tissue when a threaded connection between the spacer 130 and plate 120 is tightened. As illustrated in FIG. 11(f), rotation of screw 137a can cause a wedge block 139c to advance, causing tips 139b of arms 139 to rotate out of spacer 130 about pivot points 139a. However, it will be appreciated that any suitable deployment mechanism can be used for arms 139.

An opening lateral femoral and a closing medial femoral osteotomy plate system may be provided in accordance with the invention that is similar to the opening and closing tibial osteotomy systems, with the exception that the plates used match the anatomy of the distal femur 3. The osteotomy system for the distal femur 3 may use the same retractors 10, 30, breakaway pins 40, spreader 90, locking head screws 200, opening wedge spacers 130, guide blocks 50 or 60, and guide bar-and-block 70 or 80 as with the tibial system.

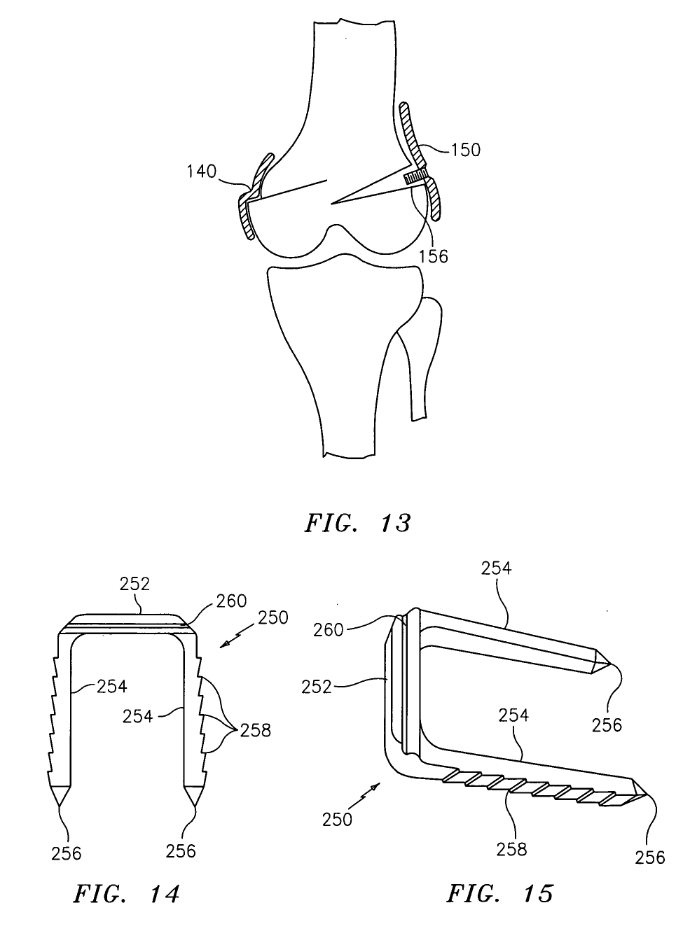

For purposes of illustration only, the overall shape of the femur plates 140, 150 is illustrated in FIGS. 12-13. The plates 140, 150 are shaped according to the shape of the distal femur—a relatively flat plate 150 for the lateral side of the femur and a plate 140 with a 1-15 mm offset (when viewed in vertical cross-section) for the medial side of the femur. The shape of the medial plate 140 or lateral plate 150 is designed to conform to the shape of the femoral condyle to which it attaches, with a staggered locking screw arrangement both above and below the level of the tenon 156.

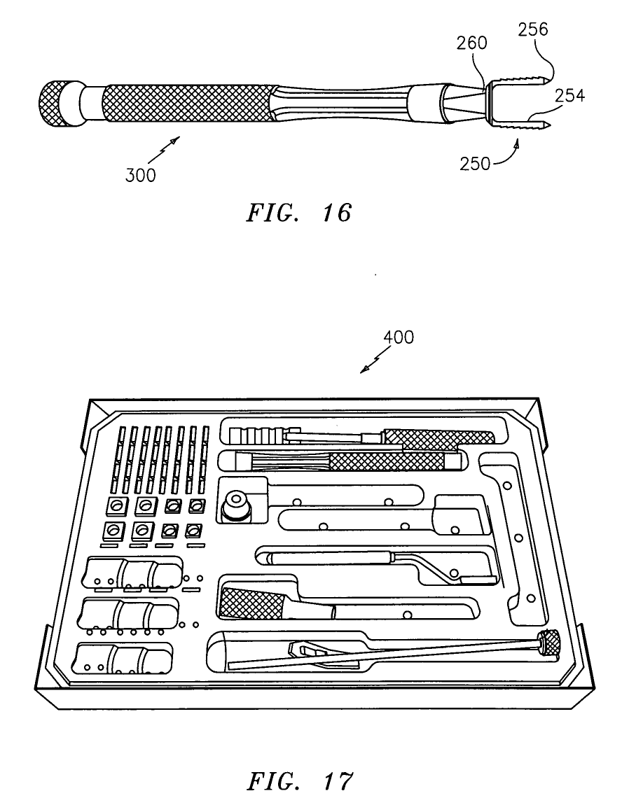

As mentioned above, fasteners such as staples 250 may be used to help hold various portions of bony anatomy in alignment during the procedures described herein. An exemplary embodiment of a bone staple is depicted in FIGS. 14-15. As depicted, staple 250 includes a main body portion 252 and two elongate prongs 254 terminating in piercing points 256. If desired, ridges or barbs 258 may be provided on prongs 254 to prevent undesired backout of the staple 250. Moreover, a gripping surface, such as recess 260 may be provided to be gripped by an insertion instrument. For purposes of illustration, such a gripping instrument 300 may be provided as depicted in FIG. 16. Instrument is adapted and configured to grip staple 250 proximate recess 260. Any suitable staples and instruments may be provided, including two, three or four prongs, as appropriate. Multiple staples may be installed next to each other in order to hold bony segments in place next to each other. Staples 250 may be left in place at the end of the procedure, if appropriate, or may be removed. As depicted in FIG. 17, a kit 400 of staples, inserters and extractors may be provided. The specific kit 400 and staples depicted in FIGS. 14-17 are commercially available from Smith & Nephew, Inc. under product no. 21-0900.

As can be seen, as embodied herein, a kit may be provided containing all the tools necessary to correct varus and valgus deformities of the knee by way of opening or closing wedge osteotomies of the femur or tibia, as desired.

The methods and systems of the present invention, as described above and shown in the drawings, provide for a complete and self-contained set of instruments required to perform an opening or closing osteotomy on either the distal femur or proximal tibia. It will be apparent to those skilled in the art that various modifications and variations can be made in the device and method of the present invention without departing from the spirit or scope of the invention. Thus, it is intended that the present invention include modifications and variations that are within the scope of the present disclosure and equivalents.

What is claimed is

1. A multi-part implant for supporting an opening wedge osteotomy, comprising:
a) a spacer for insertion into an opening created during the osteotomy procedure, the spacer having a first contoured surface; and
b) a plate for spanning the opening created during the osteotomy procedure, the plate having:
i) a first portion for attachment to bony tissue proximate a first side of the opening;
ii) a second portion for attachment to bony tissue proximate a second side of the opening; and
iii) a third portion for attachment to the spacer, the third portion of the plate including a second contoured surface that complements the first contoured surface to provide alignment between the spacer and plate when they are attached.

2. The multi-part implant of claim 1, wherein the plate defines a length along a direction that spans an opening created during an osteotomy procedure and a width generally transverse to the length, wherein the average width of the plate is between about 1.5 cm and about 2.5 cm.

3. The multi-part implant of claim 1, wherein the plate has a width sufficient to cover at least 50% of the width of the medial surface of the tibia.

4. The multi-part implant of claim 1, wherein the plate has a width sufficient to cover at least 60% of the width of the medial surface of the tibia.

5. The multi-part implant of claim 2, wherein the plate includes two rows of alternating holes along a majority of its length.

6. The multi-part implant of claim 2, wherein the plate further includes a widened portion proximate an end of the plate adapted and configured to be attached to a head of a tibia.

7. The multi-part implant of claim 2, wherein the plate has a thickness between about three and a half millimeters and about four and a half millimeters.

8. The multi-part implant of claim 1, wherein the spacer includes at least two opposed bone engagement surfaces for engaging cortical bone created by an osteotomy.

9. The multi-part implant of claim 8, wherein the opposed bone engagement surfaces are substantially parallel.

10. The multi-part implant of claim 8, wherein the opposed bone engagement surfaces are tapered along an anterior-posterior direction.

11. The multi-part implant of claim 10, wherein the opposed bone engagement surfaces diverge along an anterior-posterior direction.

12. The multi-part implant of claim 10, wherein the opposed bone engagement surfaces converge along an anterior-posterior direction.

13. The multi-part implant of claim 10, wherein the opposed bone engagement surfaces taper with respect to each other at an angle of about five degrees.

14. The multi-part implant of claim 1, wherein the spacer defines an interior volume adapted and configured for receiving material therein.

15. The multi-part implant of claim 14, further comprising material disposed in the interior volume to facilitate growth of bony tissue therethrough.

16. The multi-part implant of claim 14, wherein the spacer is shaped and sized to fill a substantial portion of an opening created during an opening wedge osteotomy procedure.

17. The multi-part implant of claim 16, wherein the spacer is defined by an annular body surrounding a hollow core.

18. The multi-part implant of claim 16, wherein the spacer is defined by an annular body made from a first material surrounding a core made from a second material.

19. The multi-part implant of claim 18, wherein the first material includes a non-resorbable material and the second material includes a resorbable material.

20. The multi-part implant of claim 19, wherein the first material includes a material selected from the group consisting of titanium, aluminum, tantalum, a polymeric material, a composite material, and combinations thereof.

21. The multi-part implant of claim 19, wherein at least one of the first and second materials is sufficiently porous to permit the growth of bony tissue therethrough.

22. The multi-part implant of claim 1, wherein the first and second portions of the plate include protrusions to facilitate anchoring the plate to bony tissue of a patient.

23. The multi-part implant of claim 1, wherein the first and second contoured surfaces include at least one alignment feature for aligning the spacer with the plate.

24. The multi-part implant of claim 1, wherein the first and second contoured surfaces comprise a dovetailed joint.

25. The multi-part implant of claim 1, wherein the spacer includes a plurality of displaceable arms that anchor into adjacent bony tissue when a threaded connection between the spacer and plate is tightened.

26. A kit for performing an opening wedge osteotomy, comprising:
a) a plurality of spacers for insertion into an opening created during the osteotomy procedure, each spacer having a first contoured surface; and
b) a plurality of plates for spanning the opening created during the osteotomy procedure, each plate having:
i) a first portion for attachment to bony tissue proximate a first side of the opening;
ii) a second portion for attachment to bony tissue proximate a second side of the opening; and
iii) a third portion for attachment to the spacer, the third portion of the plate including a second contoured surface that complements the first contoured surface to provide alignment between the spacer and plate when they are attached.

27. The kit of claim 26, further including a plurality of screws for attaching the plates to bony tissue, each screws having at least one polished surfaces.

28. The kit of claim 26, wherein each screw has a head adapted and configured to attach to the plate.

29. The kit of claim 26, wherein at least two of the spacers are different sizes.

30. The kit of claim 26, wherein at least two of the plates are different sizes.

31. The kit of claim 26, wherein at one of the spacers is tapered along the anterior-posterior direction.

32. The kit of claim 26, further comprising at least one fastener for connecting a spacer to a plate.

33. The kit of claim 26, further comprising a cutting guide for attachment to bony tissue of a patient, the cutting guide defining a groove for receiving and guiding a bone saw.

34. The kit of claim 33, further comprising an adjustable bone spreader for spreading open a cut formed in bony tissue of a patient by a surgeon.

35. The kit of claim 26, further comprising a retractor for retracting a patient's patellar tendon including a pointed distal tip adapted and configured to be anchored into bony tissue to facilitate retraction of the patellar tendon.

36. The kit of claim 26, further including at least one staple for implantation into bony tissue opposite an opening defined in a patient's bony tissue during an opening wedge osteotomy procedure.

37. A bone plate adapted and configured for spanning a gap formed in a tibia in a patient subsequent to performing an opening wedge osteotomy, comprising a generally rectangular body having a length along a direction that spans the gap and a width generally transverse to the length, wherein the average width of the plate is between about 1.5 cm and about 2.5 cm.

38. The bone plate of claim 37, wherein the plate has a width sufficient to cover at least 50% of the width of the medial surface of a tibia of a patient.

39. The bone plate of claim 37, wherein the plate has a width sufficient to cover at least 60% of the width of the medial surface of a tibia of a patient.

40. The bone plate of claim 37, wherein the plate includes two rows of alternating holes along a majority of the length of the plate.

41. The bone plate of claim 37, wherein the plate further includes a widened portion proximate an end of the plate shaped for attachment to a head of a patient's tibia.

42. The bone plate of claim 37, wherein first and second portions of the plate include protrusions for anchoring the plate to bony tissue of a patient.

43. The bone plate of claim 37, wherein the plate further includes a spacer disposed on an anatomically-facing surface for maintaining an open wedge osteotomy.

44. The bone plate of claim 43, wherein the spacer and plate are removably attached to each other by a fastener.

45. The bone plate of claim 44, wherein each of the spacer and plate include cooperating alignment features for maintaining registration between the spacer and plate.
